(19) United States
(12) Patent Application Publication
(10) Pub. No.: US 2024/0138837 A1
(43) Pub. Date: May 2, 2024

(54) ANASTOMOTIC DEVICE

(71) Applicants: Clayton A. KAISER, Nashville, TN (US); Evelio RODRIGUEZ, Brentwood, TN (US); Leandro CURA, Montevideo (UY)

(72) Inventors: Clayton A. KAISER, Nashville, TN (US); Evelio RODRIGUEZ, Brentwood, TN (US); Leandro CURA, Montevideo (UY)

(21) Appl. No.: 17/769,346

(22) PCT Filed: Jun. 30, 2021
(86) PCT No.: PCT/US2021/039737

§ 371 Date: Apr. 15, 2022

Related U.S. Application Data
(60) Provisional application No. 63/047,885, filed on Jul. 2, 2020.

(51) Int. Cl. A61B 17/11; A61L 31/06

(52) U.S. Cl. CPC A61B 17/11; A61L 31/06; A61B 2017/1107; A61B 2017/1132

(57) ABSTRACT

A vascular connector includes a tubular sleeve graft having a first layer and a second layer and a cylindrical connector body positioned within the tubular sleeve graft between the first layer and the second layer. The cylindrical connector body is more rigid than the tubular sleeve graft and is configured to slide longitudinally within the tubular sleeve graft between the first layer and the second layer.

### CROSS REFERENCE TO RELATED APPLICATIONS

This application claims priority to U.S. Provisional Patent Application No. 63/047,885, filed on Jul. 2, 2020, titled “ANASTOMOTIC DEVICE,” the entirety of which is incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

During major open-heart operations for large vessel disease, such as ascending aortic aneurysms and dissections, a large portion of the vessel (e.g., the ascending aorta) is typically replaced with a synthetic graft. Synthetic grafts are commonly made from a flexible fabric (e.g., polyester) material, such as Dacron™. The placement of a synthetic graft in a large vessel requires that the surgeon cut and remove a diseased portion of the vessel and then connect the synthetic graft to a more normal portion of the vessel, such as by running suture of small synthetic filament (e.g., polypropylene) between the graft and the vessel. This surgical procedure is called “anastomosis.”

In cases of severe atherosclerosis and high calcium deposition within the vessel (e.g., the aorta), however, even “normal” vessel tissue (to which the synthetic graft is attached) can have significant calcium deposition, making an anastomosis with the standard technique of a running suture line difficult. In even more severe cases of vessel dissection, the wall of the vessel (e.g., the aorta) has split in two, and the remaining tissue left behind to which the graft is expected to be sewn is friable. Connecting to such friable tissue can result in a sub-optimal anastomosis and can lead to a significant amount of blood loss once the synthetic graft is pressurized by the patient's heart and normal blood circulation is resumed.

Accordingly, an anastomotic device that addresses some of all of these problems is desired.

### SUMMARY OF THE DISCLOSURE

In general, in one embodiment, a vascular connector includes a tubular sleeve graft having a first layer and a second layer and a cylindrical connector body positioned within the tubular sleeve graft between the first layer and the second layer. The cylindrical connector body is more rigid than the tubular sleeve graft and is configured to slide longitudinally within the tubular sleeve graft between the first layer and the second layer.

This and other embodiments can include one or more of the following features. The cylindrical connector body can include polyoxymethylene. The tubular sleeve graft can include polyethylene terephthalate. The tubular sleeve graft can include polytetrafluoroethylene. The vascular connector can further include a band configured to compress vascular tissue between the cylindrical connector body and the band. The cylindrical connector body can include grooves along an external surface thereof configured to enable secure placement of the band. The vascular connector can further include an additional cylindrical connector body positioned within the tubular sleeve. The tubular sleeve can include a plurality of side-branches extending therefrom. The cylindrical connector can includes a plurality of holes extending circumferentially therearound.

In general, in one embodiment, a vascular connector includes a flexible tubular graft, a semi-rigid connector body positioned within the flexible tubular graft, and a wedge configured to engage with the semi-rigid connector body to transition the semi-rigid connector body from the collapsed configuration to the expanded configuration. The semi-rigid connector body has a collapsed configuration and an expanded configuration.

This and other embodiments can include one or more of the following features. The semi-rigid connector body can include a cylindrical body having a longitudinal slit therein. The wedge can be configured to engage with the longitudinal slit. The longitudinal slit can include a serrated edge, and the wedge can include a serrated edge configured to engage with the serrated edge of the longitudinal slit so as to lock the wedge within the longitudinal slit. The semi-rigid connector body in the collapsed configuration can have a diameter of 4-6 mm, and the semi-rigid connector body in the expanded configuration can have a diameter of 6-8 mm. The semi-rigid connector body can include polyoxymethylene. Flexible tubular graft can include polyethylene terephthalate. The flexible tubular graft can include polytetrafluoroethylene. The vascular connector can further include a band configured to compress vascular tissue between the cylindrical connector body and the band. The semi-rigid connector body can include grooves along an external surface thereof configured to enable secure placement of the band. The semi-rigid connector can include a plurality of holes extending circumferentially therearound.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1C show an exemplary anastomotic device. FIG. 1A shows a circumferential cross-section of the anastomotic device. FIG. 1B shows a side perspective view of the anastomotic device. FIG. 1C shows a longitudinal cross-section of the anastomotic device.

FIGS. 2A-2B show use of an exemplary anastomotic device in a large vessel, such as an aorta. FIG. 2A shows a side perspective view. FIG. 2B shows a circumferential cross-section.

FIGS. 3A-3C show a cylindrical connector of an exemplary anastomotic device.

FIG. 3A shows a side perspective view. FIG. 3B shows a side view. FIG. 3C is a close-up of a portion of FIG. 3B.

FIG. 4 shows an anastomotic device including a sleeve with one or more cylindrical connectors therein.

FIGS. 5A-5D show an exemplary cylindrical connector having a two-part expandable design. FIG. 5A shows a perspective view of the two parts of the cylindrical connector. FIG. 5B shows the wedge of the cylindrical connector. FIG. 5C shows the cylindrical connector in a compressed configuration. FIG. 5D shows the cylindrical connector in an expanded configuration.

FIG. 6 shows another exemplary cylindrical connector having a two-part expandable design.

FIG. 7 shows an exemplary cylindrical connector with a plurality of holes for sewing attachment to a large vessel, such as an aorta.

### DETAILED DESCRIPTION

Described herein is an anastomotic device that allows for a rapid and secure connection between a synthetic graft and a native aorta or other large vessel, such as the innominate, left common carotid, or left subclavian arteries.

The anastomotic device described herein can advantageously allow for the rapid connection of a synthetic graft to the native vessel, which can reduce surgical time and thereby reduce ischemic time to vital organs (e.g., the brain). The anastomotic device can also advantageously provide a secure connection, resulting in less bleeding when blood flow is resumed to the vessel. This can be particularly important when the native vessel has reduced integrity from atherosclerosis or dissection.

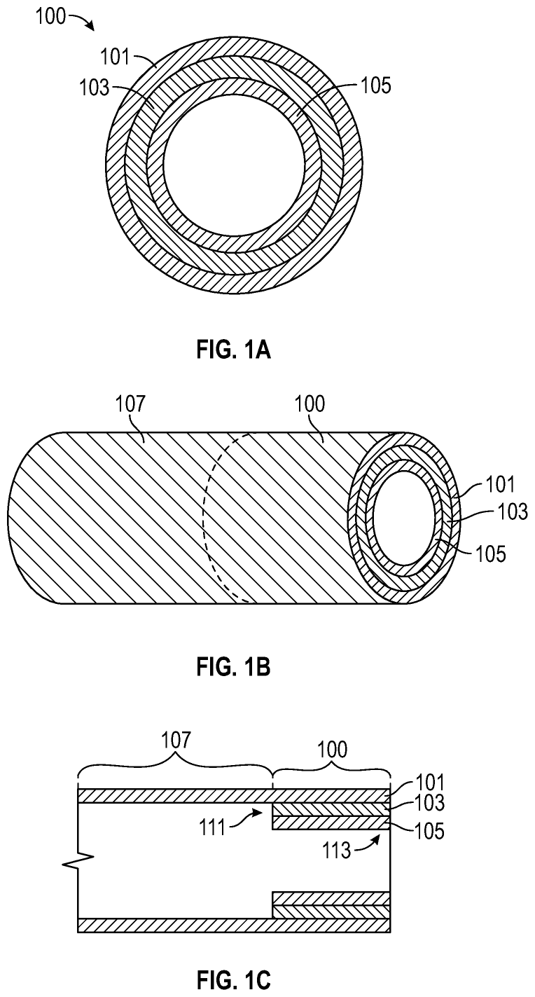

Referring to FIGS. 1A-1C, an exemplary anastomotic device 100 can include a semi-rigid cylindrical connector 103 that is wrapped with an outer layer 101 and an inner layer 105. The outer layer 101 can be made of a synthetic graft material and can be continuous with the graft 107 itself. The inner layer 105 can be substantially the same length as the cylindrical connector 103 and can also be made of a synthetic graft material. The cylindrical connector 103 can be made, for example, of a rigid plastic, such as a polyoxymethyelene homopolymer or copolymer (e.g., Delrin® or Acetal™). The inner and/or outer layers 105/101 can be made, for example, of polyethylene terephthalate (PET) or polytetrafluoroethylene (PTFE), such as Dacron™ or Gelweave™. In some embodiments, the cylindrical connector 103 can be fully covered with the synthetic graft material such that no portion of the cylindrical connector 103 comes into contact with the patient's blood. For example, the inner layer 105 and outer layer 101 can be continuous with one another and wrapped around the exposed end 113 of the device 100. The inner layer 105 can further be sealed to the inner surface of the outer layer 101 at the opposite end 111 of the device 100. In some embodiments, the anastomotic device 100 can be 20 mm-30 mm, such as 25 mm long.

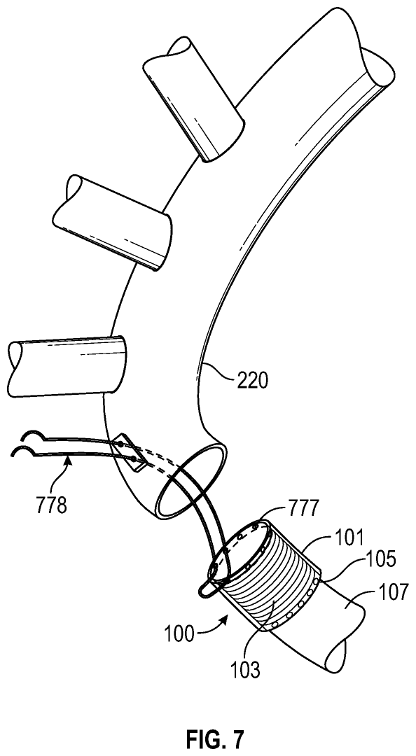

Although the graft 107 is shown and described with respect to FIGS. 1A-1C as being continuous with the outer layer 101 while the inner layer 105 is substantially the same length as the cylindrical connector 103, it should be understood that other configurations are possible. For example, as shown in FIG. 7, the graft 107 may be continuous with the inner layer 105 while the outer layer 101 may be substantially the same length as the cylindrical connector. This configuration advantageously allows the device to replace a long segment of aorta, longer than the connector device 103, while at the same time also excluding any contact of blood and/or fluid from the connector device 103.

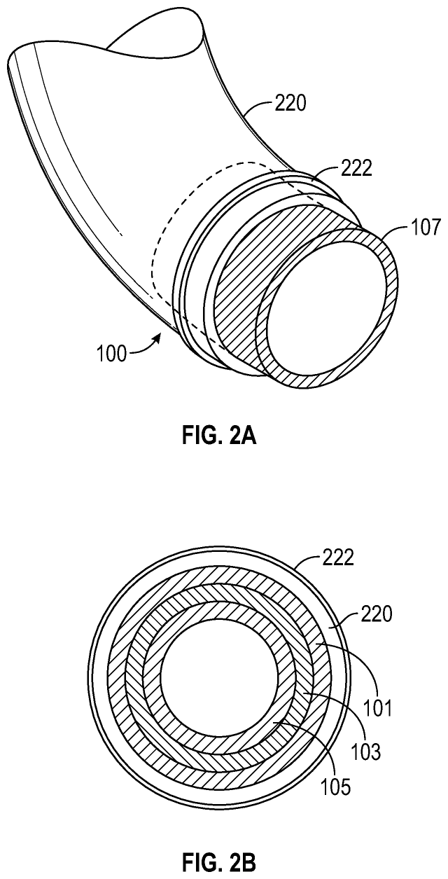

Referring to FIGS. 2A-2B, to use the device 100 in a large vessel (such as the aorta 220), the anastomotic device 100 can be placed such that the cylindrical connector 103 is within the aorta 220 and the end 111 (See FIG. 1C) substantially aligns with the severed end of the aorta 220. The graft 107 can extend distally from the severed aorta 220 and the anastomotic device 100. Further, one or more external bands 222 can be placed around the aorta 220 so as to sandwich the aorta 220 between the bands 222 and the device 100. In some embodiments, the bands 222 can be cable ties or compression bands. The external bands 222 can be made, for example, of a plastic, such as a thermoplastic, such as polyoxymethylene, such as Delrin® acetal homopolymer. Further, in some embodiments, the bands 222 can be applied with a spring-loaded applicator that is pre-set with the appropriate tension for safe and secure anastomosis.

Referring to FIG. 7, in some embodiments, the cylindrical connector 103 can further include holes 777 at one or more ends of the cylinder. There can be 2-20 holes 777, such as 4-12 holes 777 that extend circumferentially around the end of the connector 103. The holes 777 can be 0.3 mm to 2 mm in size. The holes 777 can advantageously provide a location for attachment or anchoring of the inner and/or outer layers 105/101 to the cylindrical connector 103. The holes 777 can additionally or alternatively provide a location for the anastomotic device 100 to be attached or tacked down to a specific location on the aorta 220 or other vessel, e.g., via suture 778 sewn through the holes 777 and the wall of the aorta 220. In some embodiments, the suture 778 can help reduce the need for added tension between the aorta 220 and the anastomotic device 100, as the suture 778 can fix the anastomotic device 100 in the intended position. The reduced tension can advantageously stem from the fact that the device 100 will be held in place by the sutures placed through the holes 777 and secured to the aorta 220 and will not rely solely on the tension of the external bands 222. In some embodiments, attaching the anastomotic device 100 to the aorta 220 via holes 777 can allow for more accurate placement of the bands 222.

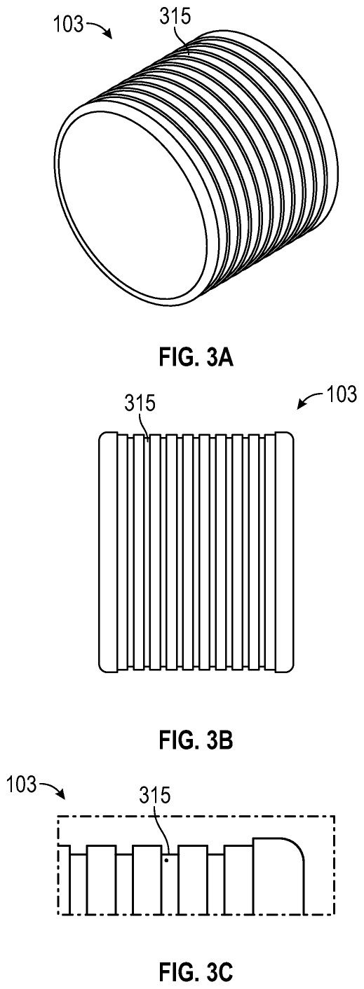

Referring to FIGS. 3A-3C, in some embodiments, the cylindrical connector 103 can include grooves 315 or indentations along the outer surface thereof to allow for secure placement of the external band(s) 222 (see FIGS. 2A-2B). For example, the grooves 315 can be 0.1 mm-0.5 mm, such as approximately 0.25 mm deep and 0.5 mm-lmm, such as 0.75 mm wide. The depth and width of the grooves 315 can be configured to enable vessel wall tissue to be sandwiched between each band 222 and groove 315 while still ensuring that the band 222 fits within the groove 315 (i.e., over the tissue and outer layer 101). The grooves 315 can advantageously prevent the band(s) 222 from slipping axially along the device 100 after securement.

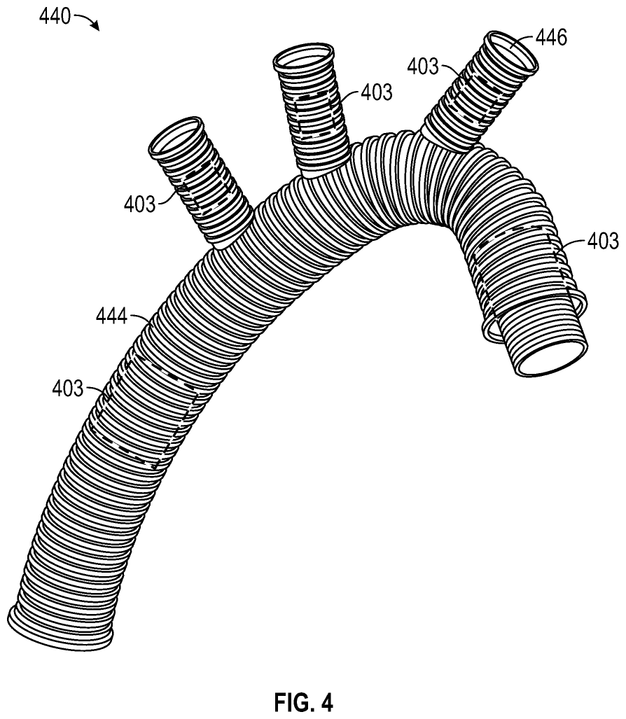

Referring to FIG. 4, in some embodiments, an anastomotic device 440 can include a cylindrical connector 403 (with features similar to connector 103) wrapped within a loose graft sleeve 444 of synthetic graft material so as to allow the cylindrical connector 403 to move therein for proper positioning of the cylindrical connector 403 within the graft sleeve 444. The cylindrical connector 403 can be moved to various locations within the graft sleeve 444 depending on the surgical application. Further, in some embodiments (and as shown in FIG. 4), the graft sleeve 444 can include a plurality of cylindrical connectors 403 therein. The portion of the graft sleeve 444 between the cylindrical connectors 403 can advantageously provide flexibility to conform to the patient's anatomy while the cylindrical connectors 403 can be used for attachment at various severed and/or weakened portions of the vessel. The adjustable positioning of a plurality of cylindrical connectors 403 can thus allow, for example, a single anastomotic device 440 to be used for the entire replacement of the ascending aorta, advantageously reducing the number of anastomoses required to complete the surgical connections. Finally, as shown in FIG. 4, in some embodiments, the graft sleeve 444 can include a plurality of side-branches 446 extending therefrom to allow for connection to branching vessels, such as the innominate, left common carotid, and/or left subclavian arteries.

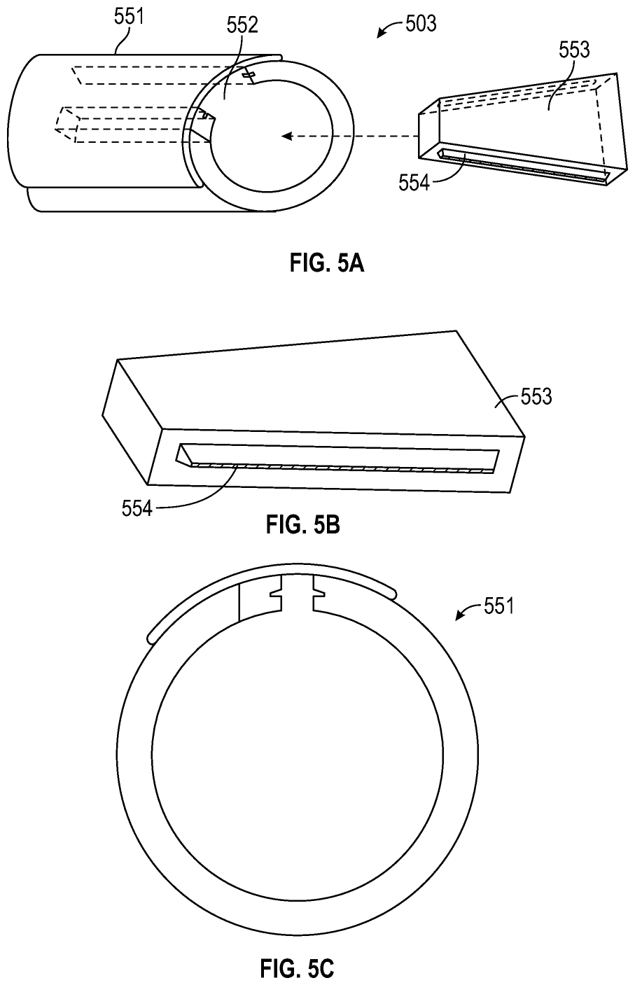

Any of the cylindrical connectors described herein can have a two-part and/or expandable design. For example, referring to FIGS. 5A-5D, the cylindrical connector 503 can include a broken cylinder 551 (i.e., a cylindrical section having a longitudinal slit 552 extending therethrough) and a wedge 553. The wedge 553 can be configured to fit within the longitudinal slit 552 to fix the cylindrical shape of the cylindrical connector 503. In some embodiments, for example, the longitudinal slit 552 can include one ore more mating edges (e.g., serrated edges) that engage with mating edges 554 (e.g., serrated edges) of the wedge 553 to as to hold the wedge 553 therein. Thus, for example, as shown in FIG. 5C, the cylindrical connector 503 can be inserted into the vessel at a compressed diameter (i.e., with the wedge 553 extending fully or partially outside of the broken cylinder 551). Once in the desired location, as shown in FIG. 5D, the wedge 553 can be inserted fully into the longitudinal slit 552, thereby causing the diameter of the broken cylinder 551 to expand such that the cylindrical connector 553 reaches its expanded and fixed diameter. The wedge 553 can be inserted by the clinician, for example, by placing pressure on the wedge 553 with a hand or finger. In some embodiments, rather than interfacing directly with the wedge 553, the clinician can place pressure on the wedge 553 through the inner and/or outer layers of the anastomotic device and onto the wedge 553. In some embodiments, the compressed diameter of the cylindrical connector 503 can be 4-6 mm while the expanded diameter can be 6-8 mm.

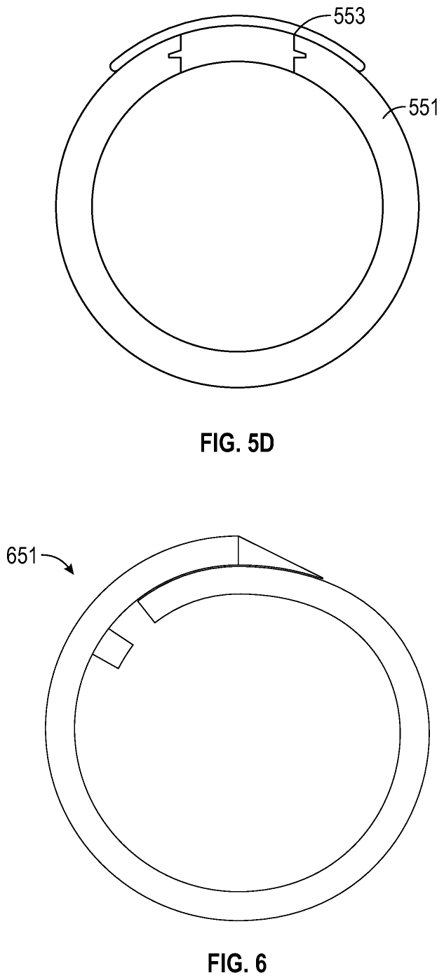

Referring to FIG. 6, in some embodiments, the broken cylinder 651 can be configured to collapse such that a portion of the broken cylinder 651 overlaps itself.

An expandable cylindrical connector as shown in FIGS. 5A-5D and 6 can advantageously facilitate easier placement of the cylindrical connector within the vessel (e.g., within the aorta or artery) while allowing expansion of the cylindrical connector within the vessel to create a larger and tighter connection. Many vessels, such as arteries, have a moderate degree of flexibility or distensibility. The expandable cylindrical connector can allow for a larger overall diameter of the connection between the anastomotic device and the native vessel, which can be better overall for increased blood flow, reduced risk of thrombosis/occlusion, and a more hemostatic connection.

In some embodiments, the devices described herein can be alternatively or additionally used for vascular connections in the abdominal aorta, including the celiac, superior mesenteric, and/or left and right renal arteries.

The devices described herein can advantageously ensure a rapid surgical anastomosis procedure that reduces surgical time and result in a more secure connection with less bleeding.

It should be understood that any feature described herein with respect to one embodiment can be used in addition to or in place of any feature described with respect to another embodiment.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A vascular connector, comprising:
a tubular sleeve graft comprising a first layer and a second layer; and
a cylindrical connector body positioned within the tubular sleeve graft between the first layer and the second layer, wherein the cylindrical connector body is more rigid than the tubular sleeve graft, and further wherein the cylindrical connector body is configured to slide longitudinally within the tubular sleeve graft between the first layer and the second layer.

2. The vascular connector of claim 1, wherein the cylindrical connector body comprises polyoxymethylene.

3. The vascular connector of claim 1, wherein the tubular sleeve graft comprises polyethylene terephthalate.

4. The vascular connector of claim 1, wherein the tubular sleeve graft comprises polytetrafluoroethylene.

5. The vascular connector of claim 1, further comprising a band configured to compress vascular tissue between the cylindrical connector body and the band.

6. The vascular connector of claim 5, wherein the cylindrical connector body comprises grooves along an external surface thereof configured to enable secure placement of the band.

7. The vascular connector of claim 1, further comprising an additional cylindrical connector body positioned within the tubular sleeve.

8. The vascular connector of claim 1, wherein the tubular sleeve comprises a plurality of side-branches extending therefrom.

9. The vascular connector of claim 1, wherein the cylindrical connector includes a plurality of holes extending circumferentially therearound.

10. A vascular connector, comprising:
a flexible tubular graft;
a semi-rigid connector body positioned within the flexible tubular graft, the semi-rigid connector body having a collapsed configuration and an expanded configuration; and
a wedge configured to engage with the semi-rigid connector body to transition the semi-rigid connector body from the collapsed configuration to the expanded configuration.

11. The vascular connector of claim 10, wherein the semi-rigid connector body comprises a cylindrical body having a longitudinal slit therein, and wherein the wedge is configured to engage with the longitudinal slit.

12. The vascular connector of claim 11, wherein the longitudinal slit comprises a serrated edge and the wedge comprises a serrated edge, the serrated edge of the wedge configured to engage with the serrated edge of the longitudinal slit so as to lock the wedge within the longitudinal slit.

13. The vascular connector of claim 10, wherein the semi-rigid connector body in the collapsed configuration has a diameter of 4-6 mm, and the semi-rigid connector body in the expanded configuration has a diameter of 6-8 mm.

14. The vascular connector of claim 10, wherein the semi-rigid connector body comprises polyoxymethylene.

15. The vascular connector of claim 10, wherein flexible tubular graft comprises polyethylene terephthalate.

16. The vascular connector of claim 10, wherein the flexible tubular graft comprises polytetrafluoroethylene.

17. The vascular connector of claim 10, further comprising a band configured to compress vascular tissue between the cylindrical connector body and the band.

18. The vascular connector of claim 17, wherein the semi-rigid connector body comprises grooves along an external surface thereof configured to enable secure placement of the band.

19. The vascular connector of claim 10, wherein the semi-rigid connector includes a plurality of holes extending circumferentially therearound.
